(19) United States
(12) Patent Application Publication
(10) Pub. No.: US 2022/0189579 A1
(43) Pub. Date: Jun. 16, 2022

(54) PROTEIN COMPLEX STRUCTURE PREDICTION FROM CRYO-ELECTRON MICROSCOPY (CRYO-EM) DENSITY MAPS

(71) Applicant: University of Washington, Seattle, WA (US)

(72) Inventors: Dong Si, Seattle, WA (US); Jonas Pfab, Seattle, WA (US); Nhut Minh Phan, Seattle, WA (US)

(73) Assignee: University of Washington, Seattle, WA (US)

(21) Appl. No.: 17/548,342

(22) Filed: Dec. 10, 2021

Related U.S. Application Data
(60) Provisional application No. 63/125,297, filed on Dec. 14, 2020.

(51) Int. Cl. G16B 15/00; G16B 40/20; G16B 30/10; G01N 1/42

(52) U.S. Cl. CPC G16B 15/00; G01N 1/42; G16B 30/10; G16B 40/20

(57) ABSTRACT

In some embodiments, a method of determining a molecular structure of a protein is provided. A computing system receives voxel data representing electron density obtained via cryo-electron microscopy. The computing system uses one or more neural networks to predict one or more likelihoods for each voxel. The computing system determines a backbone structure based on the predicted likelihoods. The computing system maps amino acid sequences to the backbone structure based on the predicted likelihoods. Mapping the amino acid sequences to the backbone structure based on the predicted likelihoods includes conducting an alignment technique that uses a reward function and a gap penalty. The computing system determines locations of carbon, nitrogen, and oxygen atoms within the backbone structure based on the predicted likelihoods. The computing system determines side-chain atoms based on the predicted backbone structure and the amino acid sequences to complete the molecular structure.

### CROSS-REFERENCE TO RELATED APPLICATION

This application claims the benefit of Provisional Application No. 63/125,297, filed Dec. 14, 2020, the entire disclosure of which is hereby incorporated by reference herein for all purposes.

### STATEMENT OF GOVERNMENT LICENSE RIGHTS

This invention was made with government support under Grant No. 2030381, awarded by the National Science Foundation. The government has certain rights in the invention.

### BACKGROUND

The determining factor for a protein's functionality is its structure, which is given by a sequence of amino acids and the sequence's three-dimensional arrangement. Consequently, researchers can draw conclusions about the behavior of the protein based on its molecular structure. This outcome can be useful in developing new vaccines and drugs, as viral fusion proteins play a central role in how the viruses invade the host's cells. In order to prevent infections, researchers attempt to develop vaccines and medicines that target these fusion proteins. This strategy is currently applied to find an effective vaccine for many viruses, including but not limited to the SARS-CoV-2 virus. The structural information about the fusion proteins is useful for researchers to predict their behaviors and ultimately to find the right vaccine.

To determine the structure of a protein, cryo-electron microscopy (cryo-EM) data may be used. Cryo-EM allows researchers to capture macromolecules' three-dimensional maps, which describe the density of electrons at a near-atomic resolution. The technology has gained popularity in recent years as an alternative to other structure determination methods, such as X-ray crystallography, due to its improved quality and efficiency. Amid the current global crisis related to SARS-CoV-2, it is significant that cryo-EM is being deployed right alongside X-ray crystallography to support the search for medicines and vaccines to fight the COVID-19 pandemic.

To derive the structure of a protein based on its 3D cryo-EM electron density map, researchers currently either have to manually fit the atoms or resort to existing template-based or homology modeling methods. The manual fitting of atoms represents an enormous manual effort as protein complexes usually consist of several thousand atoms, making it virtually impossible for larger structures. Therefore, there is a tremendous demand for a method that automatically determines the molecular structure from a cryo-EM density map. Unfortunately, existing tools such as Rosetta, MAINMAST, and Phenix determine only fragments of a protein complex, or require extensive manual processing steps. Due to the ability of cryo-EM to capture multiple large proteins in the course of a single study, a fully automated, efficient tool to determine complex structures would increase the throughput of the technology and speed up the development of medicines.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In some embodiments, a method of determining a molecular structure of a protein is provided. A computing system receives voxel data representing electron density obtained via cryo-electron microscopy. The computing system uses one or more neural networks to predict one or more likelihoods for each voxel. The computing system determines a backbone structure based on the predicted likelihoods. The computing system maps amino acid sequences to the backbone structure based on the predicted likelihoods. Mapping the amino acid sequences to the backbone structure based on the predicted likelihoods includes conducting an alignment technique that uses a reward function and a gap penalty. The computing system determines locations of carbon, nitrogen, and oxygen atoms within the backbone structure based on the predicted likelihoods. The computing system determines side-chain atoms based on the predicted backbone structure and the amino acid sequences to complete the molecular structure. The computing system stores the molecular structure in a non-transitory computer-readable medium.

In some embodiments, a non-transitory computer-readable medium having computer-executable instructions stored thereon is provided. The instructions, in response to execution by one or more processors of a computing system, cause the computing system to determine a molecular structure of a protein by performing actions including receiving, by the computing system, voxel data representing electron density obtained via cryo-electron microscopy; using, by the computing system, one or more neural networks to predict one or more likelihoods for each voxel; determining, by the computing system, a backbone structure based on the predicted likelihoods; mapping, by the computing system, amino acid sequences to the backbone structure based on the predicted likelihoods, wherein mapping the amino acid sequences to the backbone structure based on the predicted likelihoods includes conducting an alignment technique that uses a reward function and a gap penalty; determining, by the computing system, locations of carbon, nitrogen, and oxygen atoms within the backbone structure based on the predicted likelihoods; determining, by the computing system, side-chain atoms based on the predicted backbone structure and the amino acid sequences to complete the molecular structure; and storing, by the computing system, the determined molecular structure.

In some embodiments, a computing system configured to perform actions for determining a molecular structure of a protein is provided. The actions include receiving, by the computing system, voxel data representing electron density obtained via cryo-electron microscopy; using, by the computing system, one or more neural networks to predict one or more likelihoods for each voxel; determining, by the computing system, a backbone structure based on the predicted likelihoods; mapping, by the computing system, amino acid sequences to the backbone structure based on the predicted likelihoods, wherein mapping the amino acid sequences to the backbone structure based on the predicted likelihoods includes conducting an alignment technique that uses a reward function and a gap penalty; determining, by the computing system, locations of carbon, nitrogen, and oxygen atoms within the backbone structure based on the predicted likelihoods; determining, by the computing system, side-chain atoms based on the predicted backbone structure and the amino acid sequences to complete the molecular structure; and storing, by the computing system, the molecular structure in a non-transitory computer-readable medium.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1A-FIG. 1B are a flowchart that illustrates an overview of a non-limiting example embodiment of a method of determining a molecular structure of a protein complex according to various aspects of the present disclosure.

FIG. 2 is a schematic drawing that illustrates a non-limiting example embodiment of the overall architecture of the neural networks used according to various aspects of the present disclosure.

FIG. 3 is an illustration of a non-limiting example embodiment of a U-net architecture for a neural network according to various aspects of the present disclosure.

FIG. 4 is a flowchart that illustrates a non-limiting example embodiment of a procedure for determining a backbone structure based on voxel data according to various aspects of the present disclosure.

FIG. 5 includes charts that illustrate aspects of a non-limiting example embodiment of an alignment technique according to various aspects of the present disclosure.

FIG. 6 is a flowchart that illustrates a non-limiting example embodiment of a procedure for determining locations of carbon, nitrogen, and oxygen atoms according to various aspects of the present disclosure.

FIG. 7A-FIG. 7C are diagrams that illustrate processing of the procedure illustrated in FIG. 6.

FIG. 8 is a block diagram that illustrates a non-limiting example embodiment of a computing device appropriate for use as a computing device with embodiments of the present disclosure.

FIG. 9A-FIG. 9D are charts that illustrate comparisons of performance between an embodiment of the present disclosure and results generated by Phenix's map-to-model function for multiple test datasets of experimental density maps, most of which depict multi-chain complexes

FIG. 10A illustrates a portion of a deposited model structure, FIG. 10B illustrates a corresponding portion of a molecular structure derived by an embodiment of the present disclosure, and FIG. 10C illustrates a corresponding portion of a molecular structure derived by Phenix.

FIG. 11A-FIG. 11D are scatter plots that compare the evaluation results for the metrics calculated by Phenix's chain comparison tool for the embodiment of the present disclosure and Phenix, for the 52 coronavirus-related density maps that have a deposited model structure.

FIG. 12 illustrates a table that includes performance results of an embodiment of the present disclosure compared against Phenix.

FIG. 13 shows structures modelled by an embodiment of the present disclosure for the EMD-30044 density map, which captures the human receptor angiotensin-converting enzyme 2 (ACE2) to which the spike protein of the SARS-CoV-2 virus binds to and the EMD-21374 density map of a SARS-CoV-2 spike glycoprotein.

FIG. 14 illustrates the comparison of the computational time used by an embodiment of the present disclosure versus Phenix.

### DETAILED DESCRIPTION

In some embodiments of the present disclosure, a fully automated software tool that can determine all-atom structures of protein complexes based on cryo-EM density maps and associated amino acid sequences is provided. In some embodiments, deep convolutional neural networks are used that allow for fast and accurate structure predictions, and post-processing of the output of the neural networks provides a final molecular structure. The techniques disclosed herein significantly improved previous methods and results.

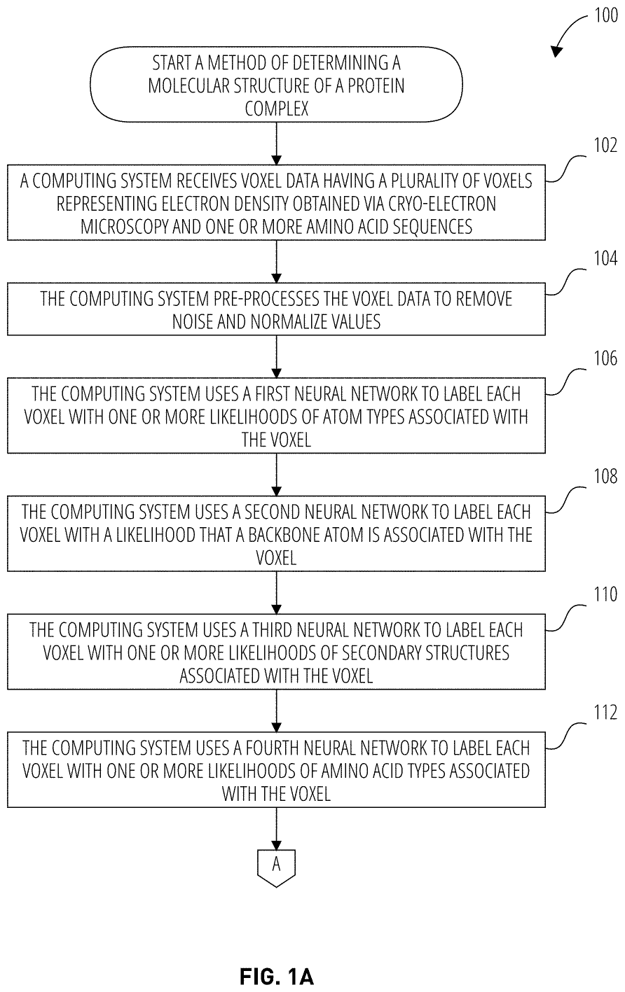

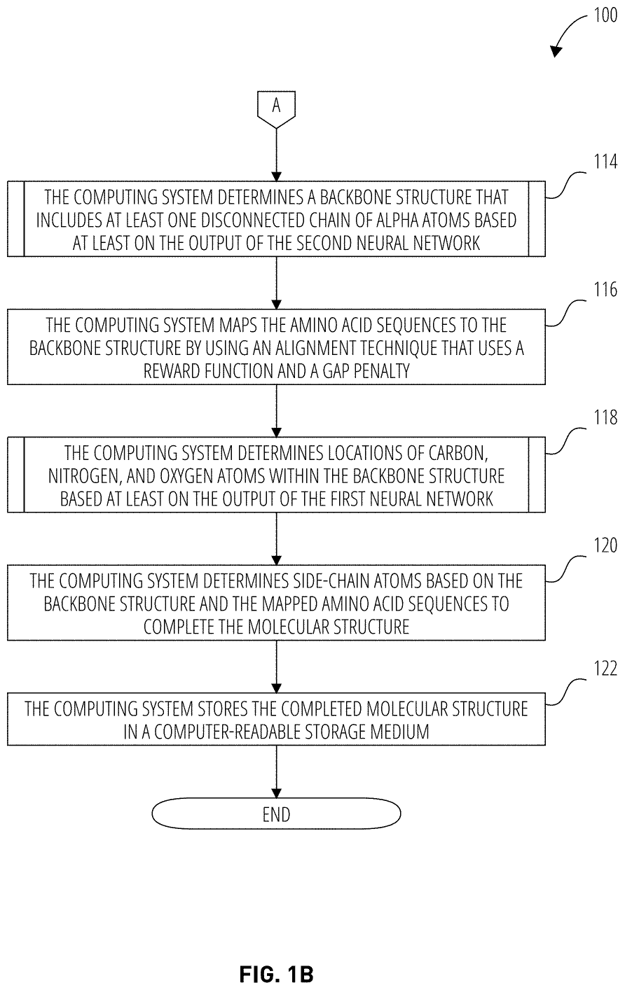

FIG. 1A-FIG. 1B are a flowchart that illustrates an overview of a non-limiting example embodiment of a method of determining a molecular structure of a protein complex according to various aspects of the present disclosure. In the method 100, cryo-EM density maps of a protein complex are analyzed by a set of deep convolutional neural networks, and the output is processed along with sequence information to automatically determine the molecular structure of the protein complex.

From a start block, the method 100 proceeds to block 102, where a computing system receives voxel data having a plurality of voxels representing electron density obtained via cryo-electron microscopy (e.g., a cryo-EM density map) and one or more amino acid sequences. The computing system may be any computing device or collection of computing devices configured to perform the actions described herein. In some embodiments, the computing system may include one or more computing devices such as desktop computing devices, server computing devices, laptop computing devices, mobile computing devices, or computing devices of a cloud computing system. In some embodiments, the computing system may use special-purpose processing devices including but not limited to graphical processing units (GPUs) and tensor processing units (TPUs) to accelerate aspects of the computations described below where appropriate.

The voxel data may be generated using any cryo-EM technique known to one of ordinary skill in the art, and may be transmitted to the computing system via a network, via a removable computer-readable storage medium, or using any other suitable technique. Likewise, the amino acid sequence information may be obtained using any sequencing technology known to one of ordinary skill in the art, and may be transmitted to the computing system using similar techniques. In some embodiments, at least one of the voxel data and the amino acid sequences may be obtained from a computer-readable storage medium, such as in cases where the voxel data and/or amino acid sequences were obtained by a third party and made publicly available for research purposes.

At block 104, the computing system pre-processes the voxel data to remove noise and normalize values. Any suitable technique or combination of techniques for denoising the voxel data may be used, including but not limited to using linear smoothing filters, anisotropic diffusion, non-local means, median filters, wavelet transforms, and statistical methods. Likewise, any suitable technique or combination of techniques for normalizing the values may be used, including but not limited to converting each value to an appropriate value between 0 and 1.

After pre-processing, the method 100 advances to blocks 106-112, where the voxel data is provided to a set of neural networks to predict four pieces of information: the locations of amino acids, the location of the backbone structure, secondary structure positions, and amino acid types. Specifically, at block 106, the computing system uses a first neural network to label each voxel with one or more likelihoods of atom types associated with the voxel; at block 108, the computing system uses a second neural network to label each voxel with a likelihood that a backbone atom is associated with the voxel; at block 110, the computing system uses a third neural network to label each voxel with one or more likelihoods of secondary structures associated with the voxel; and at block 112, the computing system uses a fourth neural network to label each voxel with one or more likelihoods of amino acid types associated with the voxel. In some embodiments, the computing system may perform the actions of blocks 106-112 in any order, and/or may perform at least some of the actions of blocks 106-112 in parallel.

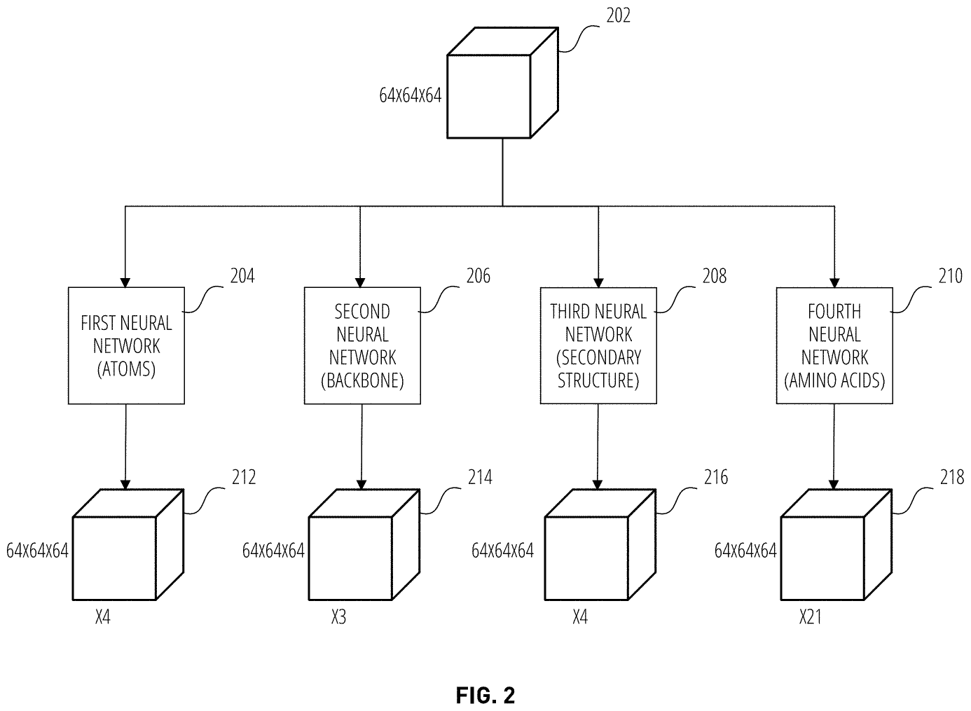

FIG. 2 is a schematic drawing that illustrates a non-limiting example embodiment of the overall architecture of the neural networks used according to various aspects of the present disclosure. As shown the pre-processed voxel data 202 is a 64³ set of three-dimensional data. The 64³ voxel data 202 is provided to a 64³ input layer of each of the first neural network 204, second neural network 206, third neural network 208, and fourth neural network 210. The output of each neural network has the same 64³ shape with a varying number of channels depending on the aspect predicted by the given neural network.

The first neural network 204 is the atoms neural network, which determines a likelihood of whether each voxel contains either a Cα atom, a nitrogen atom, a carbon atom, or no atom. Thus, the output of the first neural network 204 has an atom type likelihood 212 output with four channels. The second neural network 206 is the backbone neural network, which determines a likelihood of whether each voxel is on the backbone, a part of a side chain, or not a part of the protein. Thus, the second neural network 206 has a backbone atom likelihood 214 output with three output channels. The third neural network 208 is the secondary structure neural network, which determines a likelihood of a specific secondary structure contained in each voxel. The secondary structure likelihood 216 output by the third neural network 208 has one output channel each for loops, sheets, helices, and no structure (i.e., four output channels). The fourth neural network 210 is the amino acid type neural network, which determines a likelihood of whether each amino acid type is present for each voxel. As 20 different types of amino acids have been found in nature, the amino acid type likelihood 218 output by the fourth neural network 210 has 21 output channels, representing the amino acids plus the case in which the voxel is not part of the protein.

In some embodiments, U-Net architectures are used for each of the neural networks. A U-Net is a convolutional neural network whose name derives from the U-shape of its architecture. Neural networks that use the U-Net architecture excel in fast and precise image segmentation tasks, particularly for biomedical applications. For embodiments of the present disclosure, traditional two-dimensional U-Net architectures have been modified to process the three-dimensional density maps used herein.

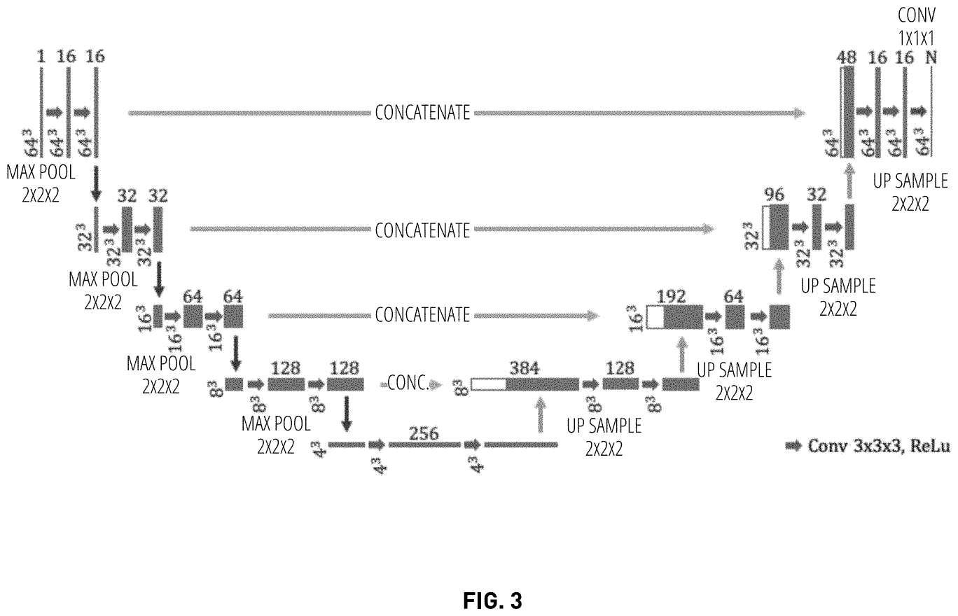

FIG. 3 is an illustration of a non-limiting example embodiment of a U-Net architecture for a neural network according to various aspects of the present disclosure. The architecture illustrated in FIG. 3 is an example of an architecture suitable for use as one or more of the neural networks illustrated in FIG. 2. In FIG. 3, the architecture includes a 64³ input layer, and a 64³ output layer with a varying number of channels depending on what structural aspect it predicts (as discussed above). As shown, a left side of the U-Net includes 3×3×3 convolutions with a ReLU function between layers and 2×2×2 max pooling to go from a higher level to a lower level. The right side of the U-Net includes 2×2×2 upsampling to go from a lower level to a higher level, concatenation from the left side of the U-Net to the right side of the U-Net, and 3×3×3 convolutions with a ReLU function between layers before a final 1×1×1 convolution to the output layer.

Before training the neural networks, a training dataset is collected. Previous projects used simulated density maps to train their neural networks. However, for the architecture to learn common noise patterns in cryo-EM density maps, it may be beneficial to use experimental density maps. In one test performed on an embodiment of the present disclosure, experimental maps were downloaded from the EM-DataResource website. Corresponding deposited model structures were retrieved from the RCSB Protein Bank and were used as ground truth data for training. To optimize the neural networks for high resolution maps, density maps with a resolution of 4 Å or better were used.

In one test embodiment, 1,800 experimental density maps and their corresponding deposited models structures were used as training data. The data was randomly split into training and validation sets with an 80:20 ratio. To label each density map, masks were created with the same dimensions as the grid of the density map, and labels were provided for each voxel based on the deposited model structures of each density map.

Separate masks were created for each of the four neural networks illustrated in FIG. 2. To train the first neural network 204 to generate atom type likelihood 212 values, an atoms mask was created that provides a label for each voxel indicating whether or not it contains a Cα, C, or N atom. To create the atoms mask, these atoms were filtered out of the corresponding model structure, corresponding grid indices for their locations were determined, and the values in the corresponding voxel and all directly neighboring voxels were set to the value representing the atom (e.g., 1 for Cα, 2 for C, and 3 for N).

Similar techniques were used to create the backbone mask, the secondary structure mask, and the amino acid mask. For the backbone mask, all backbone atoms and side-chain atoms were identified in the corresponding model structure, and the respective voxels within a distance of 2 from the identified atoms were set to 1 for backbone atoms and 2 for side-chain atoms. For the secondary structure mask, all atoms in the model structure that were part of helices, sheets, or loops were identified, and the respective voxels within a distance of 4 surrounding the atoms were set to 1 for loops, 2 for helices, and 3 for sheets. For the amino acid type mask, all Cα atoms for each of the 20 amino acid types were identified, and surrounding voxels within a distance of 3 were set to a value between 1 and 20 corresponding to the specific amino acid type.

Returning to the illustration of the method 100 in FIG. 1A, the method 100 proceeds from block 112 to a continuation terminal (“terminal A”). From terminal A (FIG. 1B), the method 100 proceeds to subroutine block 114,

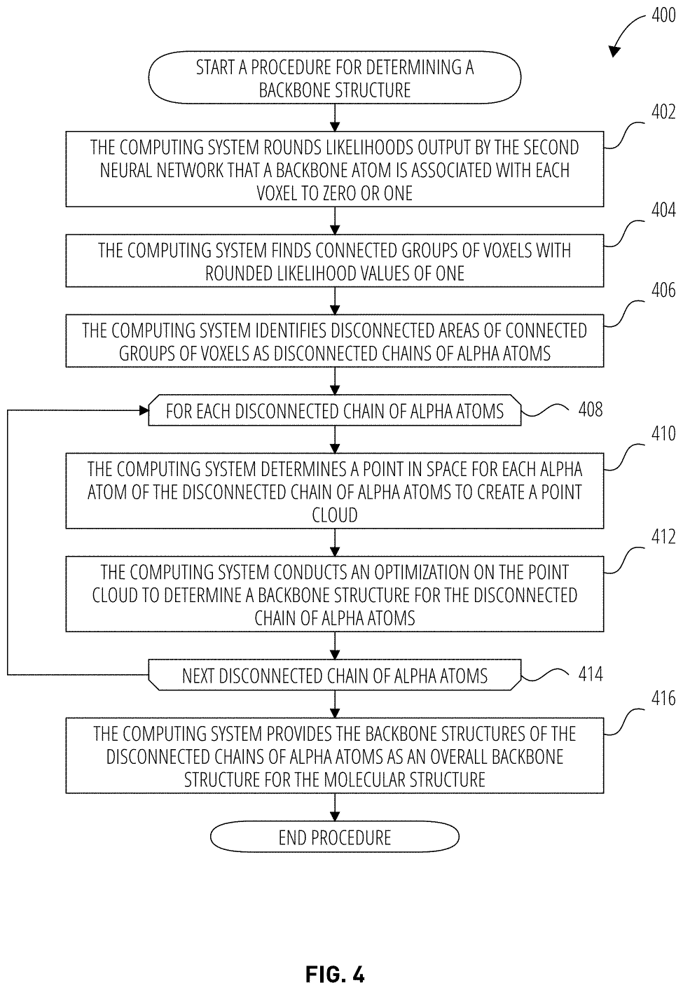

In subroutine block 114, a subroutine is executed in which the computing system determines a backbone structure that includes at least one disconnected chain of alpha atoms based at least on the output of the second neural network. Identifying backbone structure prior to any atom prediction provides several advantages. For example, performance of the backbone structure identification is improved because each chain of alpha atoms will have a smaller number of atoms to be connected via an optimization technique. As another example, using these techniques decreases the number of incorrect connections between atoms of separate chains, because they are processed independently. Any suitable subroutine for determining a backbone structure of at least one disconnected chain of alpha atoms may be used. One non-limiting example of a suitable subroutine is illustrated in FIG. 4 and described in detail below.

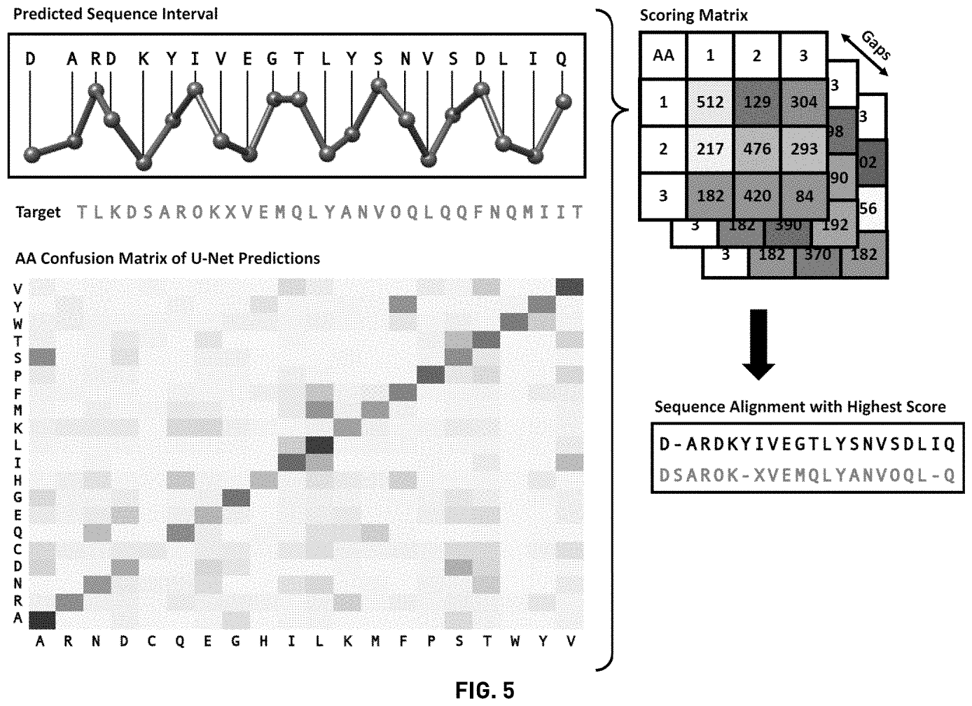

At block 116, the computing system maps the amino acid sequences to the backbone structure by using an alignment technique that uses a reward function and a gap penalty. As discussed above, an output of the fourth neural network 210 is a prediction of amino acid type likelihoods 218. However, depending on the resolution of the density map, the amino acid type likelihoods 218 generated by the fourth neural network 210 may be of limited accuracy of around 10% to 50%, since some amino acids have a similar appearance in electron density maps. Accordingly, a goal of the actions at block 116 is to improve the amino acid type identification accuracy by aligning intervals of the initially predicted sequence indicated by the amino acid type likelihood 218 to the known amino acid sequences, and then updating the types of the predicted amino acids accordingly. Further illustration and discussion of the mapping of the amino acid sequences is provided below in association with FIG. 5.

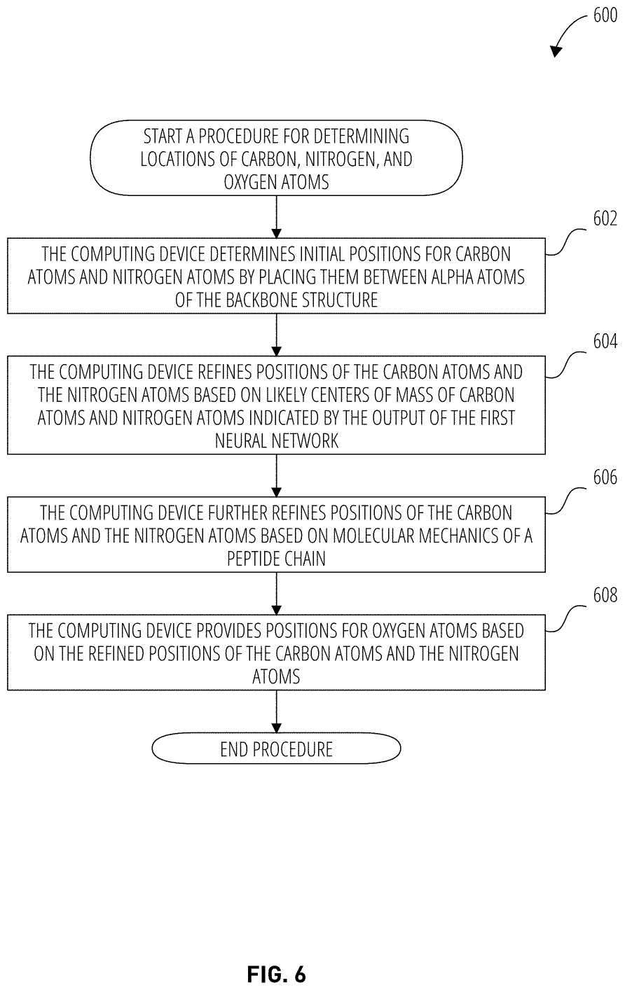

After block 116, the determined residues on the backbone structure are limited to the Cα atoms. A complete protein backbone structure also includes carbon, nitrogen, and oxygen atoms. Accordingly, at subroutine block 118, the computing system determines locations of carbon, nitrogen, and oxygen atoms within the backbone structure based at least on the output of the first neural network. Any suitable subroutine for determining the locations of the carbon, nitrogen, and oxygen atoms may be used, including but not limited to the subroutine illustrated in FIG. 6 and discussed in further detail below.

At block 120, the computing system determines side-chain atoms based on the backbone structure and the mapped amino acid sequences to complete the molecular structure. Any suitable technique for determining the side-chain atoms may be used. One non-limiting example embodiment of such a technique is to use the SCWRL4 tool developed by the Dunbrack lab, which predicts side-chain atoms for structures that have a complete backbone structure and amino acid types specified. In some embodiments, this tool may perform a collision detection to ensure that side-chains of different residues do not overlap.

After the side-chain atoms have been determined, the molecular structure has been completely determined. Accordingly, at block 122, the computing system stores the completed molecular structure in a computer-readable storage medium. The completed molecular structure may be used for any purpose, including but not limited to drug discovery. The method 100 then proceeds to an end block and terminates.

FIG. 4 is a flowchart that illustrates a non-limiting example embodiment of a procedure for determining a backbone structure based on voxel data according to various aspects of the present disclosure. The procedure 400 is a non-limiting example of a procedure suitable for use at subroutine block 114 of FIG. 1B. In the procedure 400, the output of the second neural network 206 is used to create an initial model structure that includes Cα atoms connected into one or more chains. The accuracy of the procedure 400 determines to a great extent how accurately the molecular structure will be determined. From a start block, the procedure 400 advances to block 402, where the computing system rounds likelihoods output by the second neural network 206 that a backbone atom is associated with each voxel to zero or one. In some embodiments, the output of the second neural network 206 for each voxel is a real value from zero to one for the likelihood that the voxel contains a Cα atom. In some embodiments, block 402 may round values for voxels that are greater than a threshold value, such as 0.5, up to one, and may round values for voxels that are less than the threshold value down to zero. In some embodiments, other threshold values may be used, including but not limited to 0.4 or 0.6.

At block 404, the computing system finds connected groups of voxels with rounded likelihood values of one, and at block 406, the computing system identifies disconnected areas of connected groups of voxels as disconnected chains of alpha atoms.

The procedure 400 then advances to a for-loop defined between for-loop start block 408 and for-loop end block 414, wherein each disconnected chain of alpha atoms is processed separately to determine a portion of the backbone structure. By identifying disconnected chains of alpha atoms, the number of alpha atoms that have to be processed during each iteration of the for-loop is greatly reduced.

From the for-loop start block 408, the procedure 400 advances to block 410, where the computing system determines a point in space for each alpha atom of the disconnected chain of alpha atoms to create a point cloud. To find the x, y, and z coordinates of each alpha atom, the procedure 400 uses the real values generated by the second neural network 206. In some embodiments, the points in space may be determined using two steps: first, indices of all local maximums in the output of the second neural network 206 within a distance of 4 voxels that have a minimum value greater than a confidence threshold (e.g., 0.5) are determined. Second, the indices are refined by calculating a center of mass of all voxels within a distance of 4 surrounding the local maximums. The centers of mass may then be used as the points in space for the alpha atoms. This refinement may be made possible by using the real-valued outputs of the second neural network 206. The group of the points in space define a point cloud for the disconnected chain of alpha atoms.

At block 412, the computing system conducts an optimization on the point cloud to determine a backbone structure for the disconnected chain of alpha atoms. The factorial growth of the number of ways in which the atoms can be connected makes it infeasible to test all possible solutions even for a low number of atoms. Therefore, an optimization algorithm may be used. In some embodiments, a modified traveling salesman algorithm may be used. The modified traveling salesman algorithm may not match every criterion of the traveling salesman problem—the shortest possible path is not necessarily the correct one, as the ideal distance between Cα is 3.8 Å. Deviations from this value are, however, possible due to prediction inaccuracies. Additionally, it is often difficult to decide only based on the distance which atoms to connect if there are multiple possibilities with a similar distance.

To address these issues, some embodiments of the present disclosure use a traveling salesman algorithm with a custom confidence function instead of solely relying on the Euclidean distance between atoms. The confidence function returns a score between 0 and 1 which indicates a confidence that two given atoms are connected in a backbone structure. The goal of the optimization is then to find connections for the disconnected chain of alpha atoms such that the sum of all confidence scores between connected atoms is maximized.

In some embodiments, the confidence function considers two factors: the Euclidean distance between the atoms, and the average density values of voxels that lay in between the atoms on the backbone confidence map predicted by the second neural network 206. The latter factor helps ensure that connections are made along the backbone of the structure. The voxels that lay between the atoms may be found using Bresenham's algorithm. To transform these metric values to a confidence score, a probability density function p(χ, μ, σ) with a mean μ that represents the ideal metric value and a standard deviation σ may be used. The function may be normalized by dividing it by the probability density value at the mean to ensure that the function returns exactly 1 at the mean. For the Euclidean distance, a mean of 3.8 and a standard deviation of 1 may be used in some embodiments. For the average backbone confidence, a mean of 1 and a standard deviation of 0.3 may be used. These standard deviations were determined based on several rounds of testing, though in other embodiments, other values may be used for the standard deviations and means. Any suitable technique may be used to combine the two values into a single confidence score, including but not limited to simply multiplying the values together.

Since implementations of the traveling salesman algorithm are designed to minimize distances between paths, the confidence scores may be subtracted from 1 before being provided to the optimization algorithm. Further, implementations of the traveling salesman algorithm typically specify a start/end point. However, the procedure 400 does not know at which atom the disconnected chain of alpha atoms will start and/or end. Accordingly, a new atom may be added that is connected to every other atom with a confidence of 1. This atom may then be specified as the start/end, and may be removed from the actual chain of alpha atoms after the optimization is complete.

The procedure 400 then proceeds to for-loop end block 414. If any further disconnected chains of alpha atoms remain to be processed, then the procedure 400 returns to for-loop start block 408 to process the next disconnected chain of alpha atoms. Otherwise, if all of the disconnected chains of alpha atoms have been processed, then the procedure 400 advances to block 416.

At block 416, the computing system provides the backbone structures of the disconnected chains of alpha atoms as an overall backbone structure for the molecular structure. The procedure 400 then advances to an end block and terminates.

FIG. 5 includes charts that illustrate aspects of a non-limiting example embodiment of an alignment technique according to various aspects of the present disclosure. As discussed above at block 116, the prediction of amino acid types in the molecular structure can be improved by aligning the predicted amino acids to the known amino acid sequences of the molecule as obtained using sequencing techniques. Aligning amino acid sequences is a common problem in the field of bioinformatics, and previous research has led to the development of multiple alignment algorithms. However, previous alignment techniques are usually applied between different proteins to measure their sequence similarities, which does not fit the use case of the present disclosure. One drawback of the previous techniques is that they typically treat all matches and mismatches in the same way, whereas embodiments of the present disclosure would be improved by treating some matches and mismatches in different ways. This stems from the fact that some amino acid types have a more similar appearance in density maps than others, which leads to some mismatches of the fourth neural network 210 being more likely than others.

To analyze the relative frequency of a certain match of predicted and true amino acid types, the fourth neural network 210 was applied to 200 different density maps, and the predicted amino acid types were compared to the actual types from the deposited model structures. The chart in the lower left of FIG. 5 is a confusion matrix formatted as a heat map that shows the comparison of the actual amino acid types with the predicted amino acid types, with darker colors indicating higher numbers of matches. As expected, the most frequent matches are those of the same predicted and true amino acid types. However, one can also see that the fourth neural network 210 often confuses some types (e.g., ALA and SER) and struggles more with other types (e.g., CYS).

To incorporate the fourth neural network 210 prediction behavior into the alignment technique, a reward function r is defined which returns a score denoting how valuable a certain match of predicted type p and true type t is. With f(p, t) defined as the relative frequency of a match, the reward function is:

r(tₚ, tₜ)=100×(f(p, t)−0. 05)

The constant 100 as a multiplier is used to balance the match rewards with gap penalties described below, and was chosen based on multiple rounds of testing. In some embodiments, a different constant may be used as the multiplier, including but not limited to other constants in the range of 90 to 110. The 0.05 constant was chosen because it represents the likelihood of a correct match if the amino acid type is chosen randomly, since there are 20 different amino acid types considered by the technique. The score is zero if the relative frequency equals this random likelihood.

In addition to the match reward described above, the alignment technique also uses a gap penalty. A gap represents a skipped amino acid in either the predicted or true sequence. This penalty, however, cannot simply be a static value because not all gaps are the same. For example, gaps in the beginning of a sequence before any matches were made should not result in any penalties as we only match short intervals of the predicted sequence, meaning it is highly unlikely that they align at the first amino acid of the true sequence. Additionally, the number of consecutive gaps is important. Cases where embodiments of the present disclosure miss an amino acid or predicts an extra amino acid appear relatively frequently, meaning that a single gap is not unlikely. However, two missed amino acids in a row is very uncommon, and three gaps in a row virtually never happens. Therefore, the penalty function p may be defined as below. The constants 20 and 30 were chosen for c₃ and c₄, respectively, based on test runs to create a good balance with the rewards function, but in some embodiments, other constants may be used, including but not limited to constants between 18 and 22 for the first value and constants between 27 and 33 for the second value.

$$p ( g , i ) - \{ \begin{matrix}0 , & if i = 0 \\ ∞ , & if g \geq 3 \\ {c}_{3} + ( g \times {c}_{4} ) , & otherwise\end{matrix}$$

Since a reward function and a penalty function have been defined, the ideal alignment can be found by maximizing the sum of all rewards and penalties using a dynamic algorithm. To do so, a recursive equation may be used which calculates the optimal solution based on an index i, which points to the current amino acid in the true sequence, an index j, which points to the current amino acid in the predicted sequence, and g, which counts the number of previous consecutive gaps. With t and p as the true and predicted sequence, the following recursive equation may be used. Any suitable approach may be used to find the solution, including but not limited to the dynamic programming “bottom-up” approach.

$$OPT ( i , j , g ) = \{ \begin{matrix}0 , & if i = 0 or j = 0 or g \geq 3 \\ max \{ \begin{matrix}OPT ( i - 1 , j - 1 , 0 ) + r ( {t}_{i} , {p}_{j} ) , \\ OPT ( i , j - 1 , g + 1 ) + p ( g , i ) , \\ OPT ( i - 1 , j , g + 1 ) + p ( g , j )\end{matrix} \} , & otherwise\end{matrix}$$

FIG. 6 is a flowchart that illustrates a non-limiting example embodiment of a procedure for determining locations of carbon, nitrogen, and oxygen atoms according to various aspects of the present disclosure. The procedure 600 is a non-limiting example of a procedure suitable for use at subroutine block 118 in FIG. 1B. While previous research has introduced various methods for reconstruction of a protein backbone from a reduced representation (such as one that only contains Cα atoms), the procedure 600 makes use of the likelihood information extracted from the 3D cryo-EM density maps to improve performance.

From a start block, the procedure 600 advances to block 602, where the computing device determines initial positions for carbon atoms and nitrogen atoms by placing them between alpha atoms of the backbone structure. In addition to the Cα likelihood predictions generated by the second neural network 206, the first neural network 204 provides likelihood information for (non-alpha) carbon atoms and nitrogen atoms. This information can be used in combination with the previously determined backbone structure (i.e., the determined positions of the Cα atoms) to place the carbon and nitrogen atoms.

Between the Cα atoms of two connected amino acids, there is always a nitrogen atom and a carbon atom. Therefore, the procedure 600 can determine initial positions for these atoms by calculating the vector from one Cα atom to the next in the backbone structure and then placing the initial positions of the nitrogen and carbon atoms at one third and two thirds of the distance of this vector.

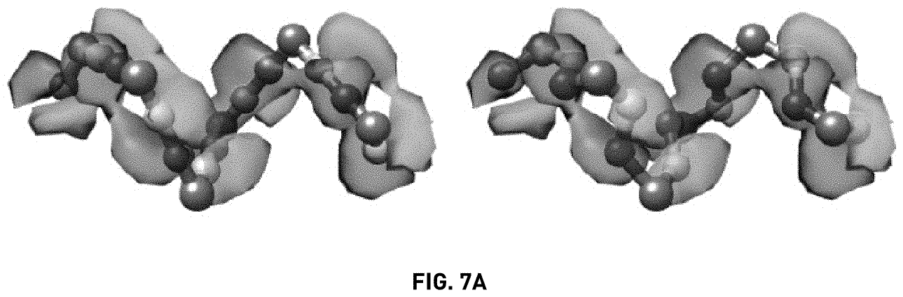

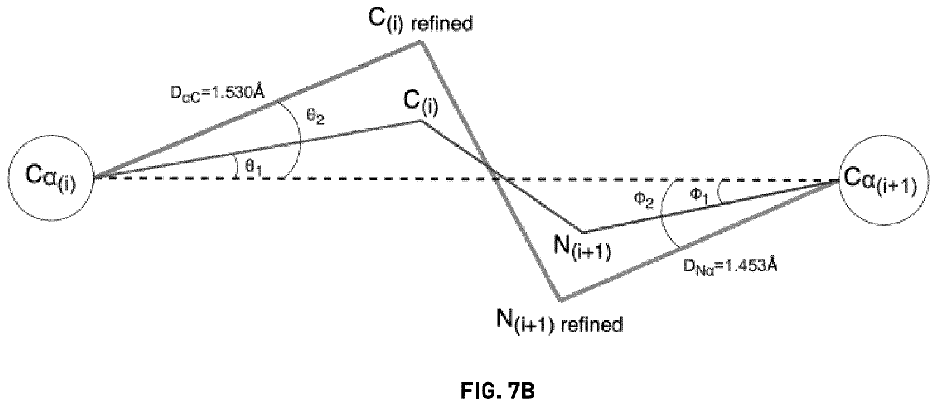

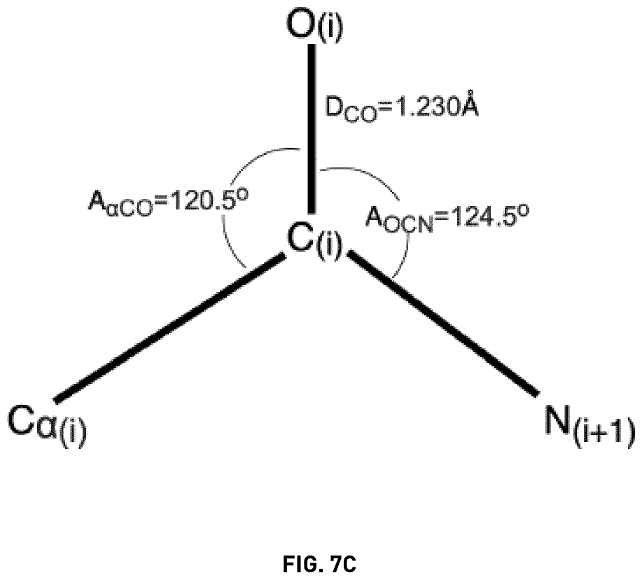

At block 604, the computing device refines positions of the carbon atoms and the nitrogen atoms based on likely centers of mass of carbon atoms and nitrogen atoms indicated by the output of the first neural network. FIG. 7A illustrates a non-limiting example for the initial and refined placements of the carbon and nitrogen atoms.

At block 606, the computing device further refines positions of the carbon atoms and the nitrogen atoms based on molecular mechanics of a peptide chain. FIG. 7B illustrates several assumptions about the positions of carbon, nitrogen, and oxygen atoms relative to the Cα atoms. First, it is assumed that the planar peptide geometry in which the Cα atom and the carbon atom in the carbonyl group of an amino acid are in the same plane as the next amino acid's nitrogen and Cα atom. Second, a virtual bond is constructed between the neighboring Cα atoms. The angles between this bond and Cα₍ᵢ₎-C₍ᵢ₎ bond (Θ₂) and between this bond and Cαᵢ₊₁₎-N₍ᵢ₊₁₎ bond (ϕ₂) are 20.9° and 14.9°, respectively. Third, the peptide bonds in a protein are in the stable trans configuration.

To further refine the position of the carbon atoms, the previous refinement is used. The unit vector pointing from Cα₍ᵢ₎ to C_{(i)refinied} may be called v₁, the unit vector pointing from Cα₍ᵢ₎ to C₍ᵢ₎ v₂, and the unit vector pointing from Cα₍ᵢ₎ to Cα₍ᵢ₊₁₎ v₃.

v₁=<a₁, a₂, a₃>

v₂=<b₁, b₂, b₃>

v₃=<c₁, c₂, c₃>

The goal is to solve for the components of v₁. Due to the planar peptide geometry, v₁, v₂, and v₃ exist in the same plane. Thus, their triple product equals to zero.

v₁×(v₂·v₃)=0

or

a₁(b₂c₃−b₃c₂)−a₂(b₁c₃−b₃c₁)=0

From this relation and the cross product of v₁ and v₂, and that of v₂ and v₃, a system of equations can be constructed:

$$\{ \begin{matrix}{a}_{1} {b}_{1} + {a}_{2} {b}_{2} + {a}_{3} {b}_{3} = cos ( {θ}_{2} - {θ}_{1} ) \\ {a}_{1} {c}_{1} + {a}_{2} {c}_{2} + {a}_{3} {c}_{3} = cos ( {θ}_{2} ) \\ {a}_{1} ( {b}_{2} {c}_{3} - {b}_{3} {c}_{2} ) - {a}_{2} ( {b}_{1} {c}_{3} - {b}_{3} {c}_{1} ) + {a}_{2} ( {b}_{1} {c}_{2} - {b}_{2} {c}_{1} ) = 0\end{matrix}$$

Solving this system of equations yields a₁, a₂, and a₃. Next, the vector vi is scaled appropriately to resolve the new position of the carbon atom. The position of the nitrogen atom is refined in a similar manner. To determine the location of the oxygen atom in the carbonyl group, a coplanar relationship between the oxygen, Cα, carbon, and nitrogen atoms is assumed, and it is also assumed that the angle A_{αCO} and A_{OCN} are approximately identical, as shown in FIG. 7C. A unit vector pointing in the direction of the C—O bond may be derived and scaled with the C—O bond length to determine the position of the oxygen atom.

In block 608, the computing device provides positions for oxygen atoms based on the refined positions of the carbon atoms and the nitrogen atoms.

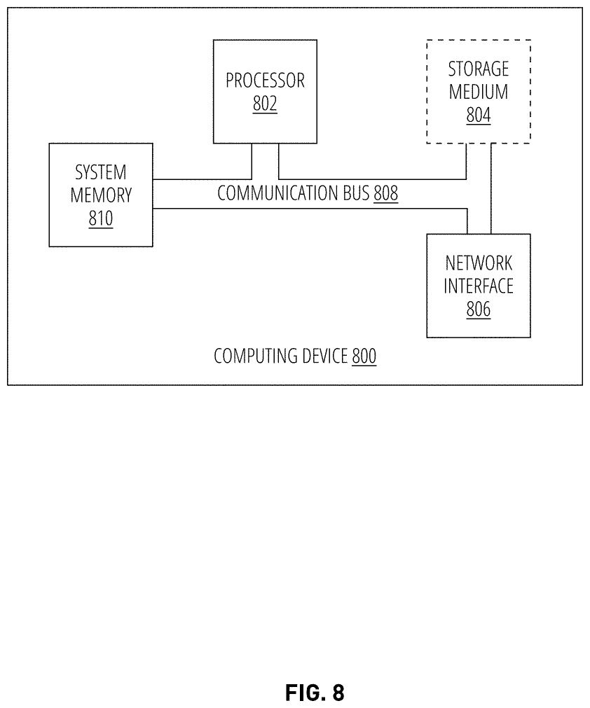

FIG. 8 is a block diagram that illustrates aspects of an exemplary computing device 800 appropriate for use as a computing device of the present disclosure. While multiple different types of computing devices were discussed above, the exemplary computing device 800 describes various elements that are common to many different types of computing devices. While FIG. 8 is described with reference to a computing device that is implemented as a device on a network, the description below is applicable to servers, personal computers, mobile phones, smart phones, tablet computers, embedded computing devices, and other devices that may be used to implement portions of embodiments of the present disclosure. Some embodiments of a computing device may be implemented in or may include an application-specific integrated circuit (ASIC), a field-programmable gate array (FPGA), or other customized device. Moreover, those of ordinary skill in the art and others will recognize that the computing device 800 may be any one of any number of currently available or yet to be developed devices.

In its most basic configuration, the computing device 800 includes at least one processor 802 and a system memory 810 connected by a communication bus 808. Depending on the exact configuration and type of device, the system memory 810 may be volatile or nonvolatile memory, such as read only memory (“ROM”), random access memory (“RAM”), EEPROM, flash memory, or similar memory technology. Those of ordinary skill in the art and others will recognize that system memory 810 typically stores data and/or program modules that are immediately accessible to and/or currently being operated on by the processor 802. In this regard, the processor 802 may serve as a computational center of the computing device 800 by supporting the execution of instructions.

As further illustrated in FIG. 8, the computing device 800 may include a network interface 806 comprising one or more components for communicating with other devices over a network. Embodiments of the present disclosure may access basic services that utilize the network interface 806 to perform communications using common network protocols. The network interface 806 may also include a wireless network interface configured to communicate via one or more wireless communication protocols, such as Wi-Fi, 2G, 3G, LTE, WiMAX, Bluetooth, Bluetooth low energy, and/or the like. As will be appreciated by one of ordinary skill in the art, the network interface 806 illustrated in FIG. 8 may represent one or more wireless interfaces or physical communication interfaces described and illustrated above with respect to particular components of the computing device 800.

In the exemplary embodiment depicted in FIG. 8, the computing device 800 also includes a storage medium 804. However, services may be accessed using a computing device that does not include means for persisting data to a local storage medium. Therefore, the storage medium 804 depicted in FIG. 8 is represented with a dashed line to indicate that the storage medium 804 is optional. In any event, the storage medium 804 may be volatile or nonvolatile, removable or nonremovable, implemented using any technology capable of storing information such as, but not limited to, a hard drive, solid state drive, CD ROM, DVD, or other disk storage, magnetic cassettes, magnetic tape, magnetic disk storage, and/or the like.

Suitable implementations of computing devices that include a processor 802, system memory 810, communication bus 808, storage medium 804, and network interface 806 are known and commercially available. For ease of illustration and because it is not important for an understanding of the claimed subject matter, FIG. 8 does not show some of the typical components of many computing devices. In this regard, the computing device 800 may include input devices, such as a keyboard, keypad, mouse, microphone, touch input device, touch screen, tablet, and/or the like. Such input devices may be coupled to the computing device 800 by wired or wireless connections including RF, infrared, serial, parallel, Bluetooth, Bluetooth low energy, USB, or other suitable connections protocols using wireless or physical connections. Similarly, the computing device 800 may also include output devices such as a display, speakers, printer, etc. Since these devices are well known in the art, they are not illustrated or described further herein.

### Results

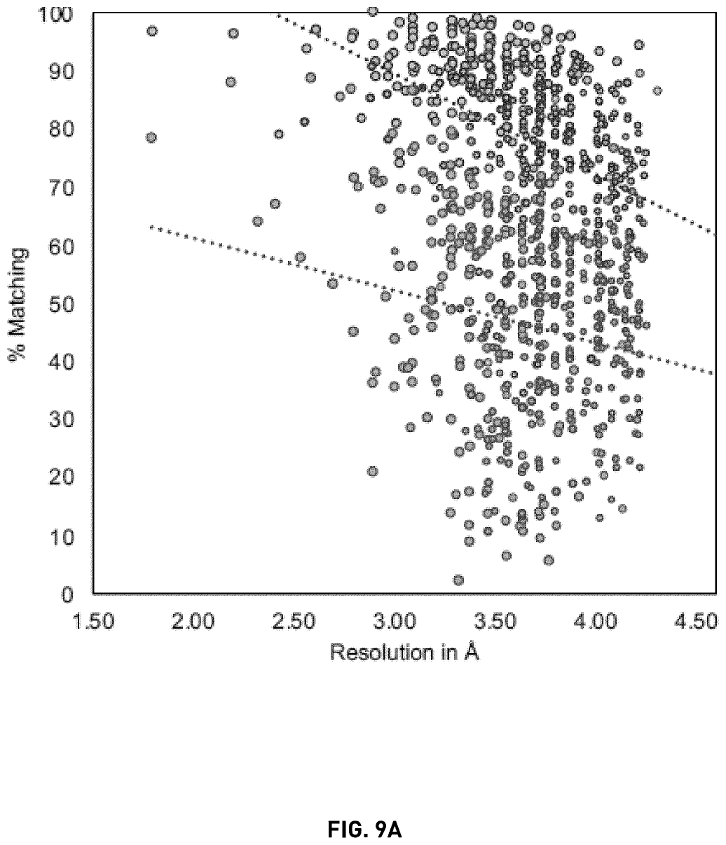

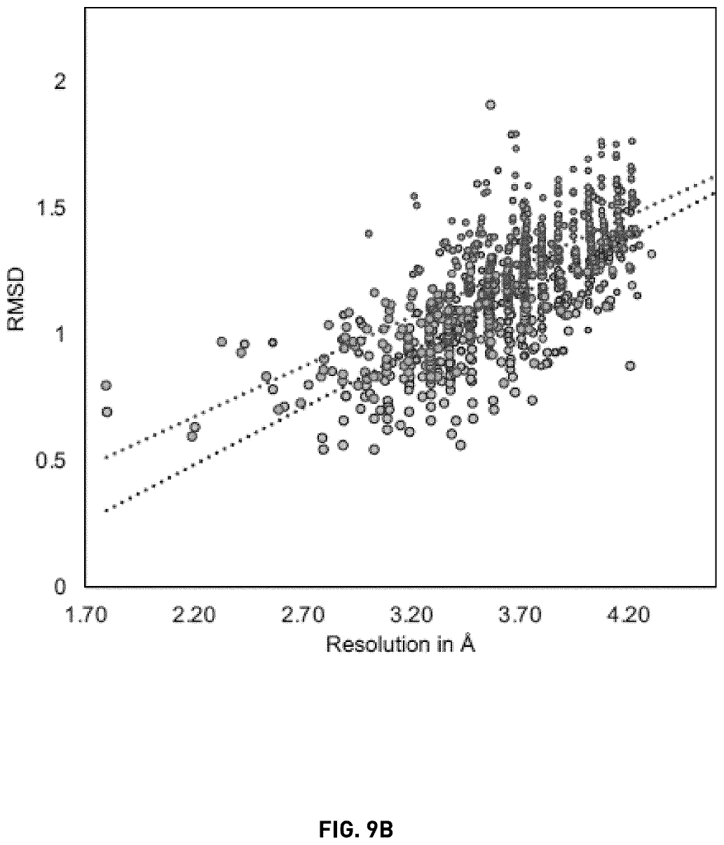

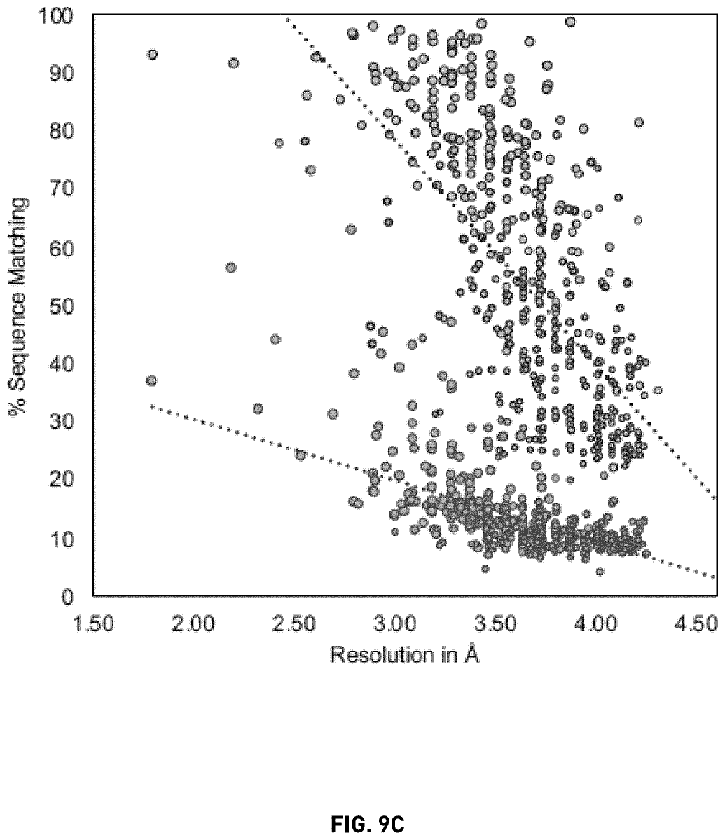

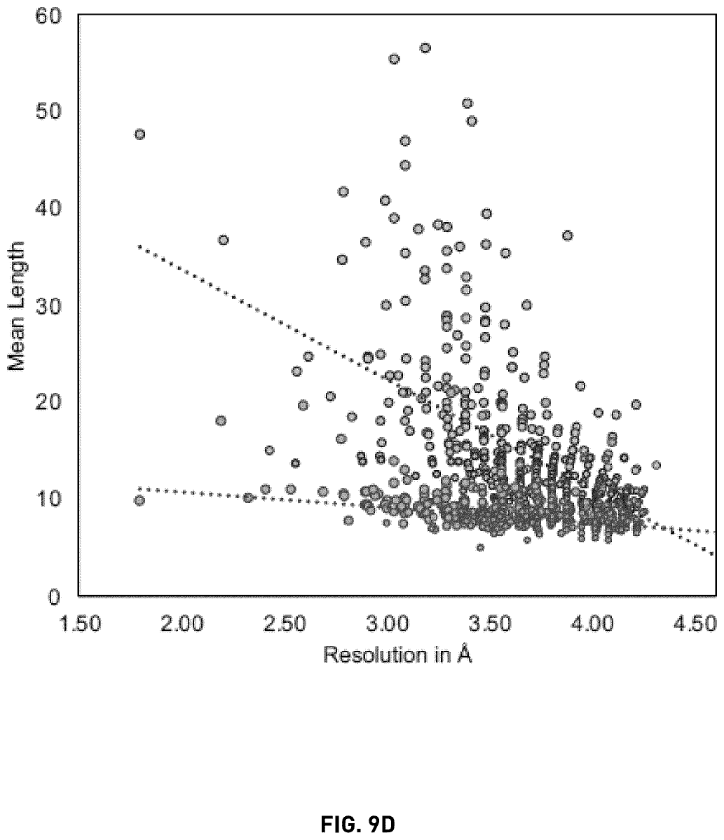

FIG. 9A-FIG. 9D are charts that illustrate comparisons of performance between an embodiment of the present disclosure and results generated by Phenix's map-to-model function for multiple test datasets of experimental density maps, most of which depict multi-chain complexes.

To ensure the objectivity of the comparison with the existing Phenix method, the phenix.chain comparison tool was used, which is available at no cost as part of the Phenix software suite. This tool compares two models by finding a one-to-one matching between their residues based on Cα positions. For two residues to match, they cannot be further apart from the other than 3 Å. Based on this matching, several metrics are calculated.

The first metric is the root-mean-square deviation (RMSD), which expresses the average distance between Cα atoms of matched residues. Second, the coverage is expressed using the matching percentage. This value represents the proportion of residues form the deposited model which have a matching interpreted residue and is calculated by dividing the number of matches by the total number of residues. Third, to evaluate how well the amino acid types were predicted, the chain comparison tool calculates the sequence matching percentage, which denotes the percentage of matched residues that have the same amino acid type. Lastly, to get a sense of how similar residues are connected, the mean length of matched segments is calculated where consecutive matches are connected in both models.

Besides the metrics calculated by the phenix.chain comparison tool, the Local-Global Alignment (LGA) algorithm is also applied, which aligns two models and computes the Global Distance Calculation (GDC) score. This score measures the similarity of two structures based on all atoms (including side-chains) on a range of 0 to 100, with 100 being a perfect match. This score was applied on the most important dataset of SARS-CoV-2 density maps due to the high manual and computational effort involved in the computation of this metric.

The embodiment of the present disclosure was applied to a set of 476 density maps assembled by the authors of Phenix's map-to-model method and the determined models were compared against the ones published on Phenix's website. It was determined that the present disclosure achieves better results than the Phenix method for every metric calculated by the phenix.chain comparison too. The matching percentage of deposited model residues is, on average, 76.93% compared to 45.65% with Phenix, representing an improvement of over 30% (see FIG. 9A). The embodiment of the present disclosure achieved a matching percentage above 70% for almost all density maps, except a few outliers.

The average RMSD value of the embodiment of the present disclosure (1.29) is 0.11 higher than that of Phenix (1.18, see FIG. 9B). One can see that the distribution of the RMSD values of the embodiment of the present disclosure follows a similar pattern as Phenix, with a strong correlation between RMSD and the resolution of the density map.

The most significant improvements of the embodiment of the present disclosure over Phenix were measured for the sequence matching, which expresses the percentage of matched residues in the determined and deposited model that have the same amino acid type (see FIG. 9C). For this metric, the embodiment of the present disclosure achieved 49.83%, which is more than four time higher than the 12.29% of the Phenix method. Although 49.83% is still fairly low, the distribution of the values shows that there is a steep improvement of the sequence matching with more accurate/higher resolution maps.

Two factors contribute to this trend. First, side chain atoms (which determine the amino acid type) are only visible in very high resolution maps, making it almost impossible to accurately predict the amino acid type for lower resolutions. Second, the amino acid type mapping of every segment can either be correct or incorrect. It means that either all amino acid types will be correct for this segment, or in case of an incorrect mapping, the amino acid types are entirely random. This amplifies the steep incline in accuracy for higher resolution maps. Third, for the last evaluated metric, the mean length of matched segments, improved from 8.16 with Phenix to 14.05 with the embodiment of the present disclosure (see FIG. 9D). While this number is influenced by several factors including the average length of connected segments in the deposited model structure, this is an indicator that the embodiment of the present disclosure connects residues better than the Phenix method.

An embodiment of the present disclosure was also compared to Rosetta and MAINMAST on a previously published set of nine density maps. Significant RMSD improvements were observed in comparison with Rosetta (from 1.37 Å to 0.85 Å) and much more complete models in comparison with MAINMAST (coverage increase of 57%, from 36.4% to 93.4%). These results represent a significant accuracy boost, resulting in more complete protein structures without any manual pre-processing steps such as zoning or cutting of the density map using a deposited model structure.

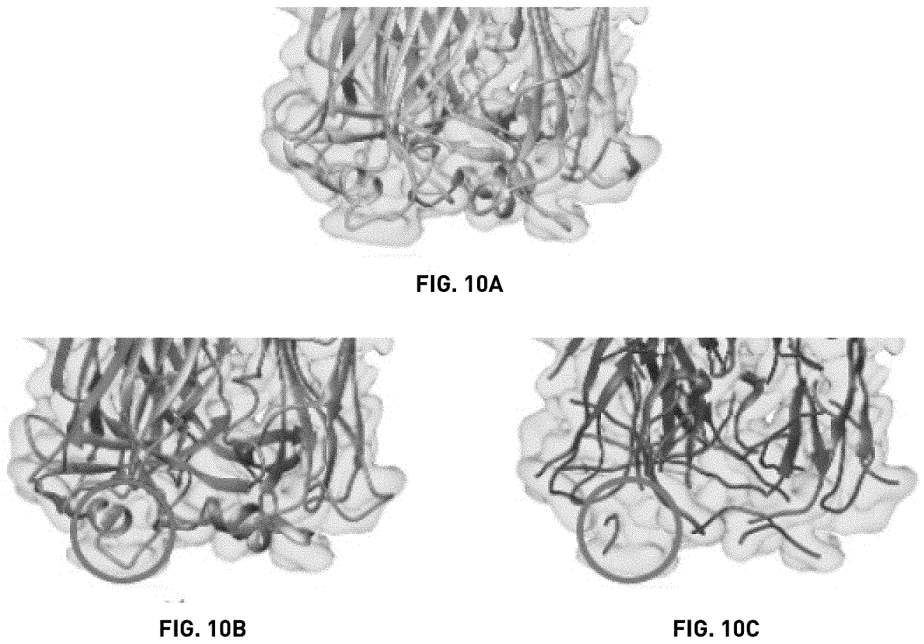

FIG. 10A illustrates a portion of a deposited model structure, FIG. 10B illustrates a corresponding portion of a molecular structure derived by an embodiment of the present disclosure, and FIG. 10C illustrates a corresponding portion of a molecular structure derived by Phenix. Each of the illustrations shows a ribbon model that highlights the backbone structure either in the deposited model or as detected by the used technique. It can be seen within the circled portion that the embodiment of the present disclosure connected the residues more completely and created a less fragmented model than Phenix.

### SARS-CoV-2 Related Results

In the search for an effective COVID-19 vaccine and medicine, structural information about the viral protein is crucial. Therefore, an embodiment of the present disclosure was tested on a set of coronavirus-related density maps to demonstrate how it can aid researchers in obtaining such structural information. To create a point of comparison, Phenix was used on the same set of density maps. The dataset was aggregated by the EMDataResource and contained 62 high-resolution density maps, 52 of which have a deposited model PDB structure. To our knowledge, this is the first CoV-related 3D cryo-EM modeling test dataset.

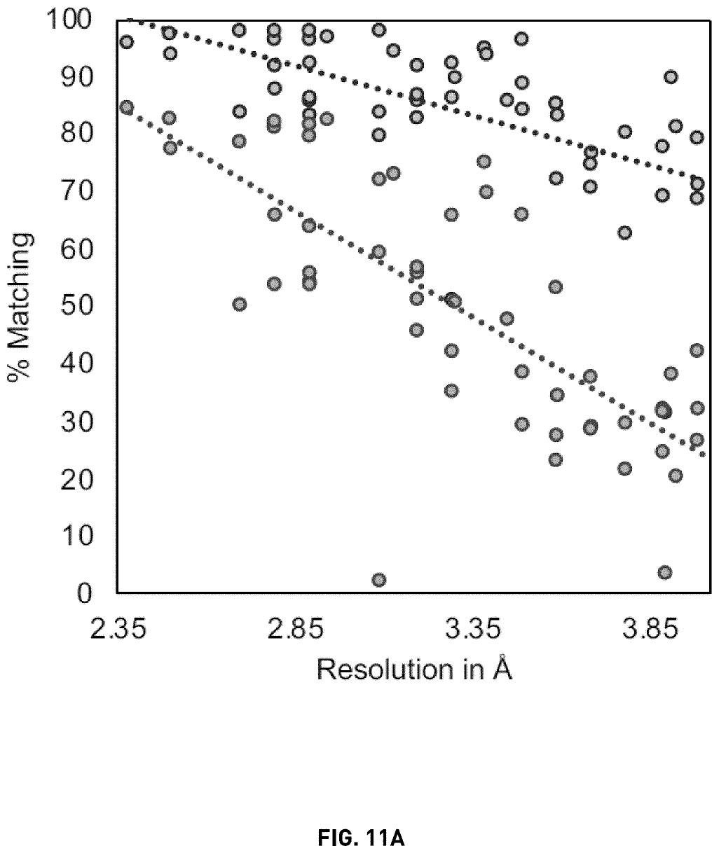

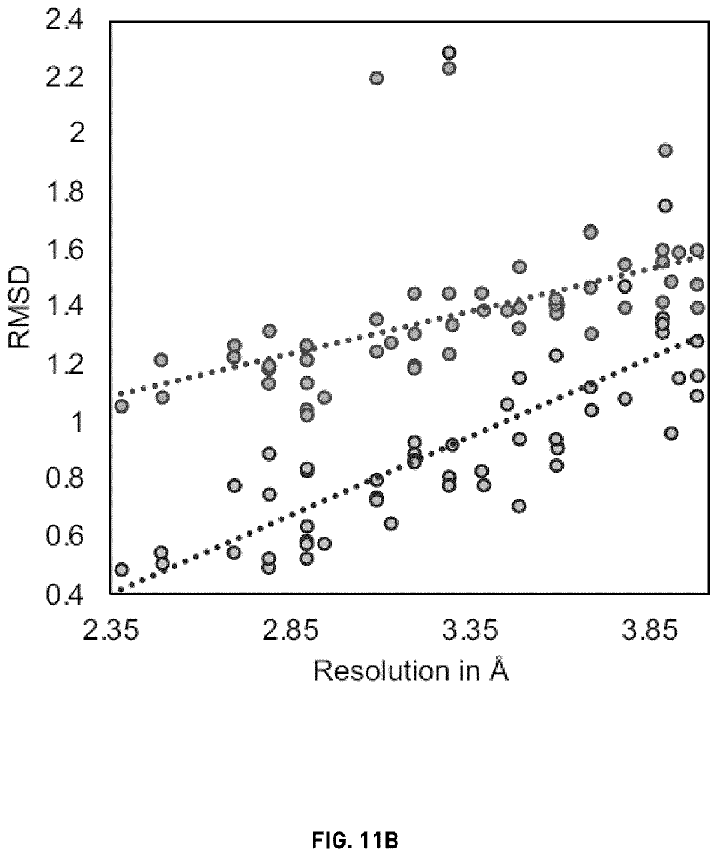

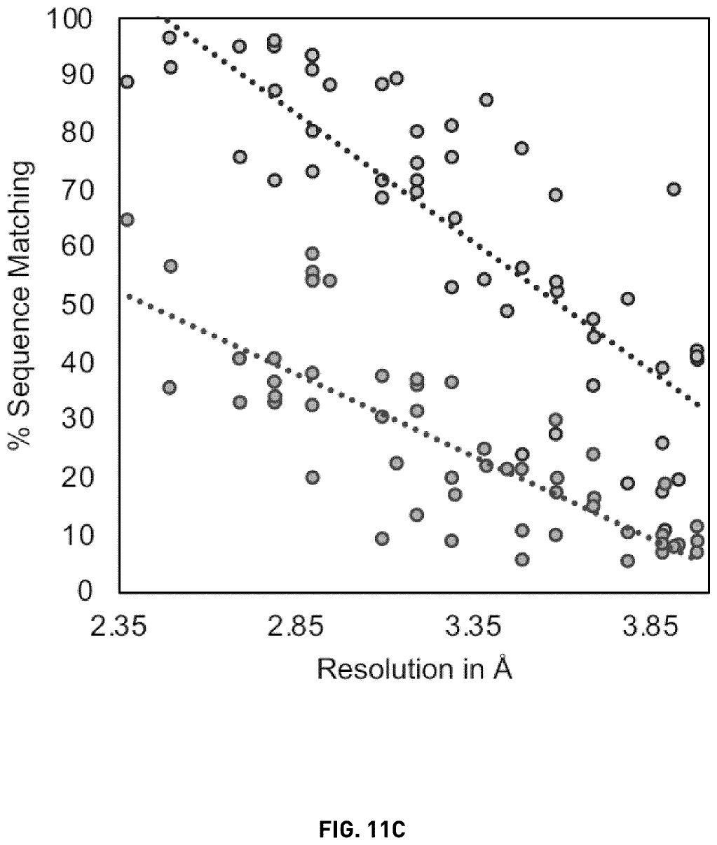

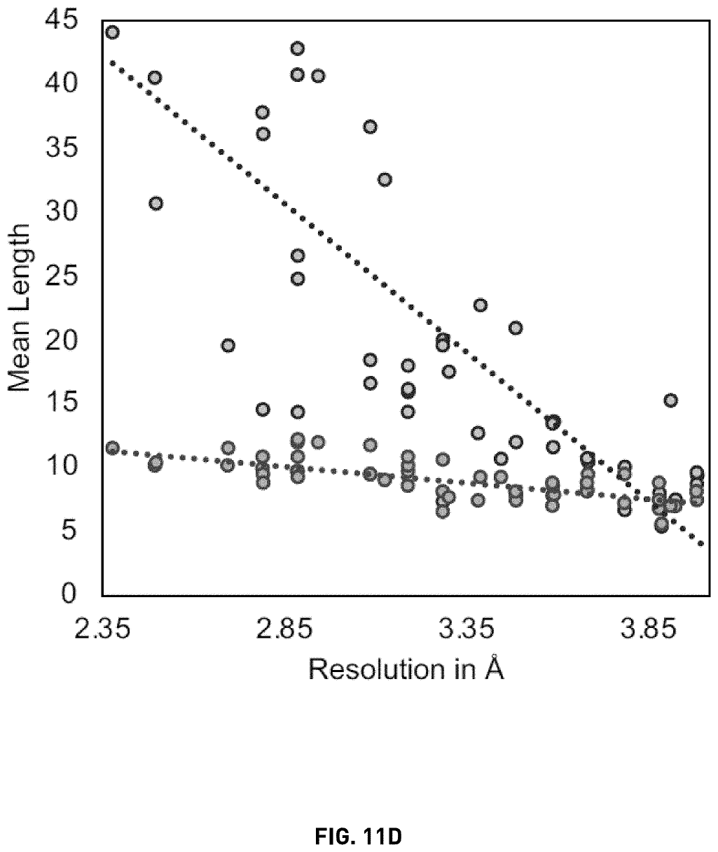

FIG. 11A-FIG. 11D are scatter plots that compare the evaluation results for the metrics calculated by Phenix's chain comparison tool for the embodiment of the present disclosure and Phenix, for the 52 coronavirus-related density maps that have a deposited model structure.

The average percentage of matched model residues is 84% for the embodiment of the present disclosure and 49.8% for Phenix (FIG. 11A). This means that, on average, around 34% more residues were correctly placed by the embodiment of the present disclosure than by Phenix.

The RMSD metric calculated an average value of 1.37 Å for Phenix compared to 0.93 Å with the embodiment of the present disclosure (FIG. 11B). Thus, the embodiment of the present disclosure not only determined more residues correctly than Phenix, but the correctly determined residues were also closer to the residues of the deposited model by around 0.4 Å.

For the sequence matching results, Phenix scored 24.95%, while the embodiment of the present disclosure achieved a sequence matching percentage of 63.08% (FIG. 11C).

Finally, the mean length of consecutively matched residues in the modelled and deposited structure increased from 8.9 with Phenix to 20 with the embodiment of the present disclosure.

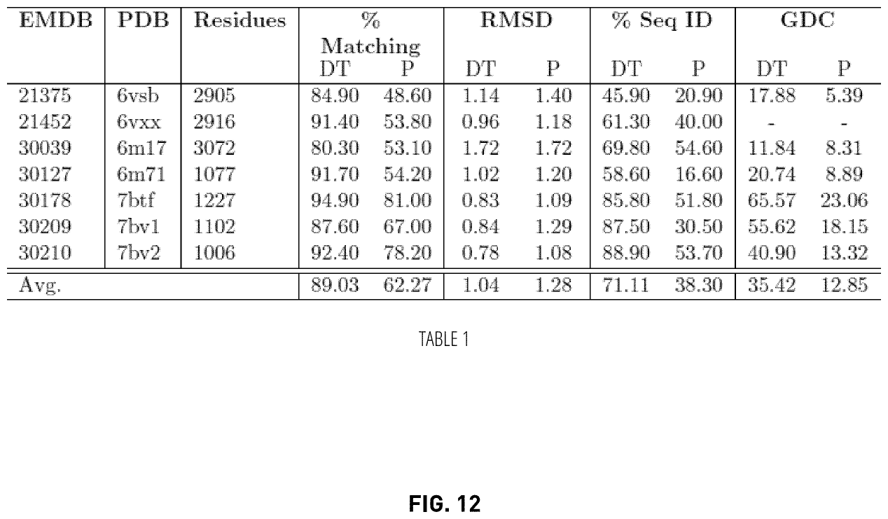

The SARS-CoV-2 results from Table 1 (FIG. 12) show a similar pattern as the results of all coronavirus-related maps. The embodiment of the present disclosure (“DT”) outperformed Phenix (“P”) in every metric with the most significant differences in the matching percentage and sequence matching. Additionally, the embodiment of the present disclosure achieved a GDC score almost three times that of the Phenix method.

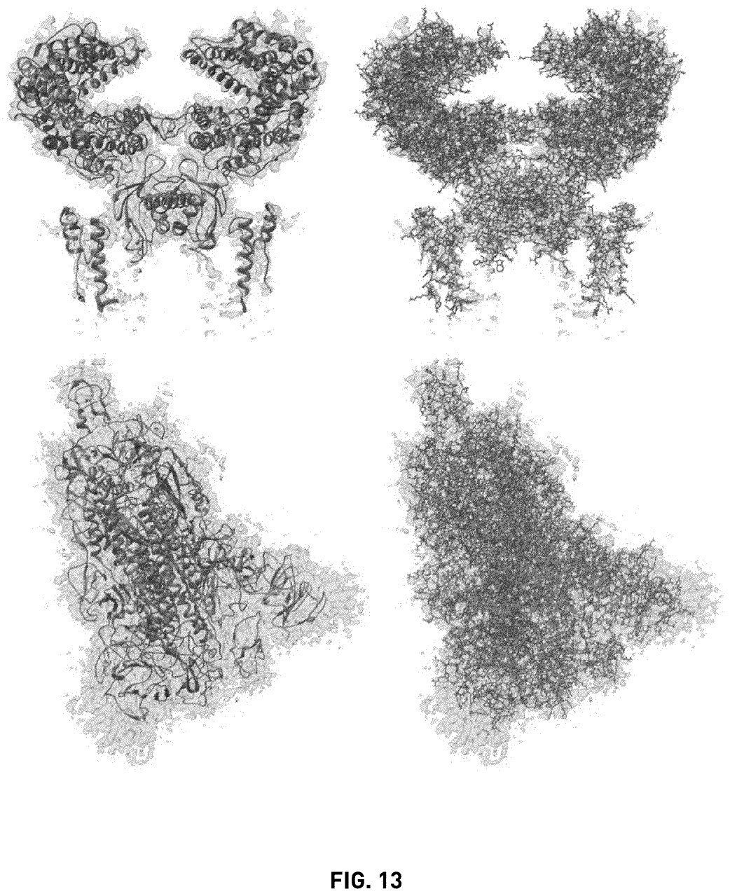

FIG. 13 shows structures modelled by an embodiment of the present disclosure for the EMD-30044 density map, which captures the human receptor angiotensin-converting enzyme 2 (ACE2) to which the spike protein of the SARS-CoV-2 virus binds to and the EMD-21374 density map of a SARS-CoV-2 spike glycoprotein. No model structure had been deposited to the EMDR for either density map as of the date this structure was generated. This represents an ideal opportunity to showcase the potential of embodiments of the present disclosure. Without any other parameters or manual processing steps, embodiments of the present disclosure can determine detailed models based on the density maps. Researchers can use these models to develop therapeutics targeting the binding process between the spike protein and the human enzyme.

### Computation Time

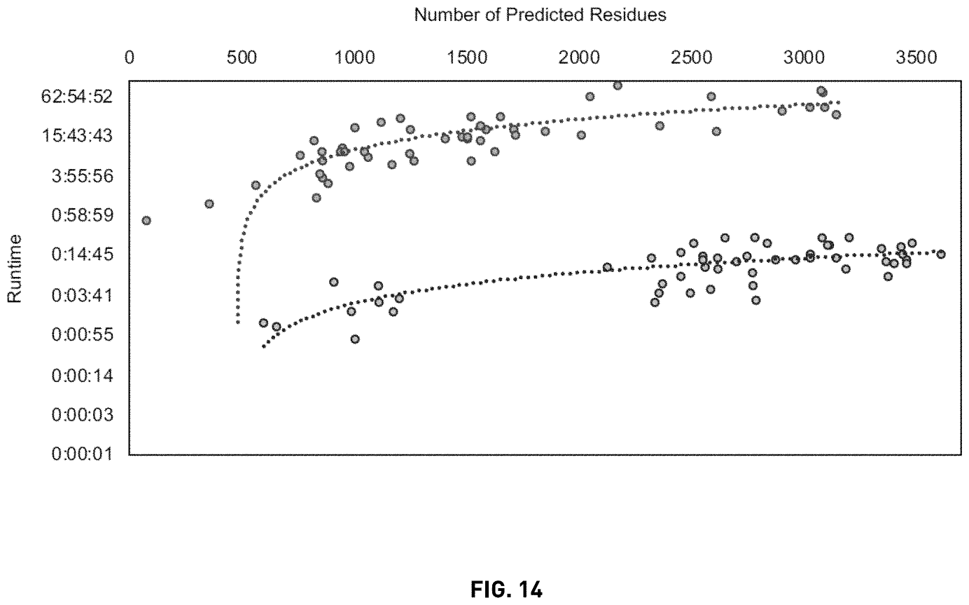

A major bottleneck of existing methods is their computational complexity, which renders them unable to model larger protein complexes. Thus, an analysis was conducted of computational time used by an embodiment of the present disclosure versus Phenix. FIG. 14 illustrates the comparison of the computational time used by an embodiment of the present disclosure versus Phenix.

The tests were executed on a machine with an Nvidia GeForce GTX 1080 Ti GPU, 8 processors, and 62 GB of memory. Although Phenix does not take advantage of the machine's GPU, this comparison provides a glimpse of the possibility that embodiments of the present disclosure can achieve. It was observed that Phenix took about 45 minutes to process a map containing 79 residues, while the embodiment of the present disclosure processed a map containing 2798 residues in only 26 minutes. Furthermore, the largest cryo-EM map (EMD-9891) that was tested required around 14 minutes to complete, whereas Phenix's processing time for this map was over 60 hours.

Embodiments of the present disclosure are able to exploit the processing power of the GPU, which is becoming a staple on modern computing systems, to increase the throughput of scientific discovery. Embodiments of the present disclosure can model even very large protein complexes in a matter of hours. As an example, it traced around 60,000 residues for the EMD-9829 density map within only two hours.

While illustrative embodiments have been illustrated and described, it will be appreciated that various changes can be made therein without departing from the spirit and scope of the invention.

The embodiments of the invention in which an exclusive property or privilege is claimed are defined as follows:

1. A method of determining a molecular structure of a protein, comprising:
receiving, by a computing system, voxel data representing electron density obtained via cryo-electron microscopy;
using, by the computing system, one or more neural networks to predict one or more likelihoods for each voxel;
determining, by the computing system, a backbone structure based on the predicted likelihoods;
mapping, by the computing system, amino acid sequences to the backbone structure based on the predicted likelihoods, wherein mapping the amino acid sequences to the backbone structure based on the predicted likelihoods includes conducting an alignment technique that uses a reward function and a gap penalty;
determining, by the computing system, locations of carbon, nitrogen, and oxygen atoms within the backbone structure based on the predicted likelihoods;
determining, by the computing system, side-chain atoms based on the predicted backbone structure and the amino acid sequences to complete the molecular structure; and
storing, by the computing system, the molecular structure in a non-transitory computer-readable medium.

2. The method of claim 1, wherein the reward function is:
r(tₚ, tₜ)=c₁×(f(p, t)−c₂)
wherein c₁ is a constant to balance the reward with the gap penalty; and
wherein c₂ is a constant that represents a likelihood of a correct match if an amino acid type is chosen randomly.

3. The method of claim 1, wherein the gap penalty is:
$$p ( g , i ) - \{ \begin{matrix}0 , & if i = 0 \\ ∞ , & if g \geq 3 \\ {c}_{3} + ( g \times {c}_{4} ) , & otherwise\end{matrix}$$
wherein i is an index of an amino acid that is not skipped, and wherein c₃ and c₄ are selected to balance the reward function.

4. The method of claim 1, wherein an ideal alignment maximizes a sum of all rewards and all penalties using a dynamic algorithm.

5. The method of claim 4, wherein using the dynamic algorithm includes applying a bottom-up approach defined as:
$$OPT ( i , j , g ) = \{ \begin{matrix}0 , & if i = 0 or j = 0 or g \geq 3 \\ max \{ \begin{matrix}OPT ( i - 1 , j - 1 , 0 ) + r ( {t}_{i} , {p}_{j} ) , \\ OPT ( i , j - 1 , g + 1 ) + p ( g , i ) , \\ OPT ( i - 1 , j , g + 1 ) + p ( g , j )\end{matrix} \} , & otherwise\end{matrix}$$
wherein i is an index of a current amino acid in the amino acid sequence,
wherein j is an index of a current amino acid in the predicted sequence, and
wherein g counts a number of previous consecutive gaps.

6. The method of claim 1, wherein using one or more neural networks to predict one or more likelihoods for each voxel includes:
providing the voxel data to a plurality of neural networks that each label each voxel with one or more predicted likelihoods, wherein the plurality of neural networks include:
a first neural network configured to label each voxel with one or more likelihoods of atom types associated with the voxel;
a second neural network configured to label each voxel with a likelihood that a backbone atom is associated with the voxel;
a third neural network configured to label each voxel with one or more likelihoods of secondary structures associated with the voxel; and
a fourth neural network configured to label each voxel with one or more likelihoods of amino acid types associated with the voxel.

7. The method of claim 6, wherein determining the locations of carbon, nitrogen, and oxygen atoms within the backbone structure based on the predicted likelihoods includes:
providing initial positions for carbon and nitrogen atoms by placing them between alpha atoms;
refining positions of the carbon and nitrogen atoms based on centers of mass of carbon atoms and nitrogen atoms in an output of the first neural network;
further refining the positions of the carbon and nitrogen atoms based on molecular mechanics of a peptide chain; and
providing positions for oxygen atoms based on the positions of the carbon and nitrogen atoms.

8. The method of claim 6, wherein determining the backbone structure based on the predicted likelihoods includes:
identifying two or more disconnected chains of alpha atoms.

9. The method of claim 8, wherein identifying two or more disconnected chains of alpha atoms includes:
rounding likelihoods output by the second neural network to zero or one;
finding connected groups of voxels with a rounded value of one for the output of the second neural network; and
identifying disconnected areas of connected groups of voxels as disconnected chains of alpha atoms.

10. The method of claim 8, wherein determining the backbone structure based on the predicted likelihoods further includes:
for each disconnected chain of alpha atoms, determining a point in space for each alpha atom of the disconnected chain of alpha atoms to create a point cloud; and
conducting an optimization on the point cloud.

11. The method of claim 10, wherein determining the point in space for each alpha atom of the disconnected chain of alpha atoms to create the point cloud includes:
finding indices of all local maximums in an output of the second neural network within a distance of 4 voxels that have a minimum likelihood value of 0.5; and
calculating a center of mass of all voxels within a distance of 4 surrounding the local maximums.

12. The method of claim 10, wherein conducting the optimization on the point cloud includes using a modified traveling salesman technique that includes scoring connections between alpha atoms using a confidence score that considers a Euclidean distance between the alpha atoms and average density values of voxels that lay in between the alpha atoms.

13. A non-transitory computer-readable medium having computer-executable instructions stored thereon that, in response to execution by one or more processors of a computing system, cause the computing system to determine a molecular structure of a protein by performing actions comprising:
receiving, by the computing system, voxel data representing electron density obtained via cryo-electron microscopy;
using, by the computing system, one or more neural networks to predict one or more likelihoods for each voxel;
determining, by the computing system, a backbone structure based on the predicted likelihoods;
mapping, by the computing system, amino acid sequences to the backbone structure based on the predicted likelihoods, wherein mapping the amino acid sequences to the backbone structure based on the predicted likelihoods includes conducting an alignment technique that uses a reward function and a gap penalty;
determining, by the computing system, locations of carbon, nitrogen, and oxygen atoms within the backbone structure based on the predicted likelihoods;
determining, by the computing system, side-chain atoms based on the predicted backbone structure and the amino acid sequences to complete the molecular structure; and
storing, by the computing system, the determined molecular structure.

14. The non-transitory computer-readable medium of claim 13, wherein the reward function is:
r(tₚ, tₜ)=c₁×(f(p, t)−c₂)
wherein c₁ is a constant to balance the reward with the gap penalty; and
wherein c₂ is a constant that represents a likelihood of a correct match if an amino acid type is chosen randomly.

15. The non-transitory computer-readable medium of claim 13, wherein the gap penalty is:
$$p ( g , i ) - \{ \begin{matrix}0 , & if i = 0 \\ ∞ , & if g \geq 3 \\ {c}_{3} + ( g \times {c}_{4} ) , & otherwise\end{matrix}$$
wherein i is an index of an amino acid that is not skipped, and wherein c₃ and c₄ are selected to balance the reward function.

16. The non-transitory computer-readable medium of claim 13, wherein an ideal alignment maximizes a sum of all rewards and all penalties using a dynamic algorithm.

17. The non-transitory computer-readable medium of claim 16, wherein using the dynamic algorithm includes applying a bottom-up approach defined as:
$$OPT ( i , j , g ) = \{ \begin{matrix}0 , & if i = 0 or j = 0 or g \geq 3 \\ max \{ \begin{matrix}OPT ( i - 1 , j - 1 , 0 ) + r ( {t}_{i} , {p}_{j} ) , \\ OPT ( i , j - 1 , g + 1 ) + p ( g , i ) , \\ OPT ( i - 1 , j , g + 1 ) + p ( g , j )\end{matrix} \} , & otherwise\end{matrix}$$
wherein i is an index of a current amino acid in the amino acid sequence,
wherein j is an index of a current amino acid in the predicted sequence, and
wherein g counts a number of previous consecutive gaps.

18. The non-transitory computer-readable medium of claim 13, wherein determining the backbone structure based on the predicted likelihoods includes:
identifying two or more disconnected chains of alpha atoms.

19. The non-transitory computer-readable medium of claim 18, wherein determining the backbone structure based on the predicted likelihoods further includes:
for each disconnected chain of alpha atoms, determining a point in space for each alpha atom of the disconnected chain of alpha atoms to create a point cloud; and
conducting an optimization on the point cloud.

20. A computing system configured to perform actions for determining a molecular structure of a protein, the actions comprising:
receiving, by the computing system, voxel data representing electron density obtained via cryo-electron microscopy;
using, by the computing system, one or more neural networks to predict one or more likelihoods for each voxel;
determining, by the computing system, a backbone structure based on the predicted likelihoods;
mapping, by the computing system, amino acid sequences to the backbone structure based on the predicted likelihoods, wherein mapping the amino acid sequences to the backbone structure based on the predicted likelihoods includes conducting an alignment technique that uses a reward function and a gap penalty;
determining, by the computing system, locations of carbon, nitrogen, and oxygen atoms within the backbone structure based on the predicted likelihoods;
determining, by the computing system, side-chain atoms based on the predicted backbone structure and the amino acid sequences to complete the molecular structure; and
storing, by the computing system, the molecular structure in a non-transitory computer-readable medium.
